(19) United States
(12) Patent Application Publication
(10) Pub. No.: US 2023/0230692 A1
(43) Pub. Date: Jul. 20, 2023

(54) SYSTEM FOR REALIZING REMOTE MONITORING OF MOVEMENT HEALTH OF PET

(71) Applicant: YUNNAN MA, KUNMING CITY (CN)

(72) Inventor: YUNNAN MA, KUNMING CITY (CN)

(21) Appl. No.: 17/673,095

(22) Filed: Feb. 16, 2022

(30) Foreign Application Priority Data
Jan. 17, 2022 (CN) 202210050413.5
Jan. 17, 2022 (CN) 202220120210.4

(51) Int. Cl. G16H 40/67

(52) U.S. Cl. CPC G16H 40/67

(57) ABSTRACT

The present invention discloses a system for achieving remote monitoring of pet movement health, comprising: an integration module, a power supply module, a remote server module and a client end, said power supply module being electrically connected to said integration module, said remote server module having signal connection to said client end. Said integration module comprises: a positioning module, a motion sensing module, a vital sign monitoring module and a communication module. Said vital sign monitoring module is electrically connected to said communication module, said vital signs monitoring module is used to acquire pet cardiorespiratory audio and detect pet heart rate and send the acquired cardiorespiratory audio information to said communication module.

### FIELD OF THE DISCLOSURE

The present invention relates to the field of pet monitoring technology, and specifically to a system for achieving remote monitoring of pet movement health.

### BACKGROUND OF THE DISCLOSURE

With the general improvement of living conditions, more and more people like to raise pets. However, due to the physiological structure of pets and the fact that pet bodies are usually covered by fur or hair, many scientific technological means cannot be directly used on pets, let alone to know about the health state of the pets in a timely manner. The commonly used pet health monitoring devices only rely on gyroscopes to determine the movement amount of the pets, which can only perform simple movement and sleep monitoring on pets without providing true information about vital signs of the pets.

Therefore, it is necessary to provide a new technical solution to solve the above shortcomings.

### SUMMARY OF THE DISCLOSURE

The goal of the present invention is to provide a system for realizing remote monitoring of pet movement health based on the shortcomings and deficiencies of the prior art, wherein the system of the present invention has a reasonable structural improvement and data collection, especially through pet movement and cardiopulmonary monitoring, realizes a real movement health monitoring function. In addition, with the improvement of the pet wearing module, the present invention fixes the smart device worn by pets, making the distant pet owner more at ease, which is advantageous for technology promotion and application.

In order to achieve the above goal, the solution of the present invention is: a system for realizing remote monitoring of the movement health of a pet used in a device worn by pets, said system for realizing remote monitoring of the movement health of a pet comprising: an integration module, a power supply module, a remote server module and a client end, said power supply module being electrically connected to said integration module, said remote server module having signal connection to said client end, characterized in that said integration module comprises: a positioning module, a motion sensing module, a vital sign monitoring module and a communication module, wherein said communication module has signal connection to said remote server module, said positioning module is electrically connected to said communication module, wherein said positioning module is used for real-time positioning and path tracking of the pet, and said positioning module is used to send the acquired pet position information and path information to said communication module, said motion sensing module is electrically connected to said communication module, wherein said motion sensing module is used to collect data information on the motion status of the pet and to compare said data information with predetermined information to determine the motion status of the pet, and said motion sensing module is also used to send the determined result to said communication module; and said vital sign monitoring module is electrically connected to said communication module, said vital signs monitoring module is used to listen to and acquire pet cardiorespiratory audio and detect pet heart rate and send the acquired cardiorespiratory audio information to said communication module, and said communication module sends said cardiorespiratory audio information to said remote server module and performs real-time analysis, and said remote server module sends the analysis results to said client end.

Said device worn by pets is further provided with a fixing member and an adjusting member, wherein said fixing member is used to fix said device on the side close to the cardiorespiratory position of the pet and said adjusting member is used to adjust the tightness of said fixing member.

Said communication module can be wired communication means or wireless communication means.

Said motion sensing module comprises: a three-axis gyroscope, wherein said three-axis gyroscope is used to obtain data information on the motion status of said pet.

Said vital sign monitoring module comprises: an audio collector, an active filter circuit, a filter interface circuit, an audio amplifier circuit, a recording circuit and an analog-to-digital conversion circuit, wherein said audio collector is used to receive pet cardiorespiratory audio information, said audio collector is electrically connected to said active filter circuit, said active filter circuit is electrically connected to said filter interface circuit, said filter interface circuit is electrically connected to said audio amplification circuit, said audio amplification circuit sends the amplified pet cardiorespiratory audio information to the analog-to-digital conversion circuit, said analog-to-digital conversion circuit sends the converted obtained pet cardiorespiratory audio information to said recording circuit, said recording circuit is electrically connected to said communication module, said communication module sends the recorded pet cardiorespiratory audio information to the remote server.

Said audio collector is coated with medical silicone.

Said audio collector is a membrane cavity pickup stethoscope head.

Said fixing member is a belt and said adjusting member is a buckle.

Said client end is a cell phone or a monitor.

Said device worn by pets is provided with an accommodation section, said accommodation section is provided with an opening, said integration module and said power supply module are provided in said accommodation section, and said opening is provided with a zipper.

With the adoption of the above technical solutions, the beneficial effects of the present invention are:

1. The system for realizing remote monitoring of the movement health of a pet comprises: an integration module, a power supply module, a remote server module and a client end, said power supply module being electrically connected to said integration module, said remote server module having signal connection to said client end, wherein said integration module comprises: a positioning module, a motion sensing module, a vital sign monitoring module and a communication module, wherein said vital sign monitoring module is electrically connected to said communication module, said vital signs monitoring module is used to listen to and acquire pet cardiorespiratory audio and detect pet heart rate and send the acquired cardiorespiratory audio information to said communication module, and said communication module sends said cardiorespiratory audio information to said remote server module and performs real-time analysis, and said remote server module sends the analysis results to said client end. The system for realizing remote monitoring of the movement health of a pet of the present invention effectively solves problems of the existing pet health monitoring equipment, which determines the amount of pet movement only by gyroscopes and can only perform simple monitoring on pet movement and sleeping and cannot truly provide feedback on the pet's vital signs. The present invention achieves a real movement health monitoring function with a reasonable structural improvement and data collection and especially through the pet movement and cardiopulmonary monitoring. In addition, with the improvement of the pet wear module, the present invention achieves a fixed smart device worn by pets, so that the distant pet owners are more at ease, which is conducive to technology promotion and application of the technology.

2. Said communication module has signal connection to said remote server module. Said positioning module is electrically connected to said communication module, wherein said positioning module is used for real-time positioning and path tracking of the pet, and said positioning module is used to send the acquired pet position information and path information to said communication module. Said motion sensing module is electrically connected to said communication module, wherein said motion sensing module is used to collect data information on the motion status of the pet and to compare said data information with predetermined information to determine the motion status of the pet, and said motion sensing module is also used to send the determined result to said communication module. This enables the positioning of the pet's location information and the recognition and sensing of the pet's movement status and trajectory.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

In order to illustrate the technical solutions in the embodiments of the present invention or of the prior art more clearly, the following is a brief description of the drawings required in the description of the embodiments or prior art, and it is obvious that the drawings in the following description are only some embodiments of the present invention, and that other drawings can be obtained from these drawings without inventive labor for those of ordinary skill in the art.

FIG. 1 is a structural block diagram of the present invention.

FIG. 2 is a schematic diagram of the structure of the vital sign monitoring module of the present invention.

FIG. 3 is a schematic diagram of the structure of the present invention.

FIG. 4 is a schematic diagram of a portion of the structure of the present invention.

### DETAILED DESCRIPTION OF THE EXEMPLARY EMBODIMENTS

The following is a further detailed description of the present invention in conjunction with the accompanying drawings.

This specific embodiment is only an explanation of the invention, and it is not a limitation of the invention. A person skilled in the art may make modifications to this embodiment as needed without inventive contribution after reading this specification, but as long as they are within the scope of the claims of the invention, they are protected by patent law.

### Embodiments

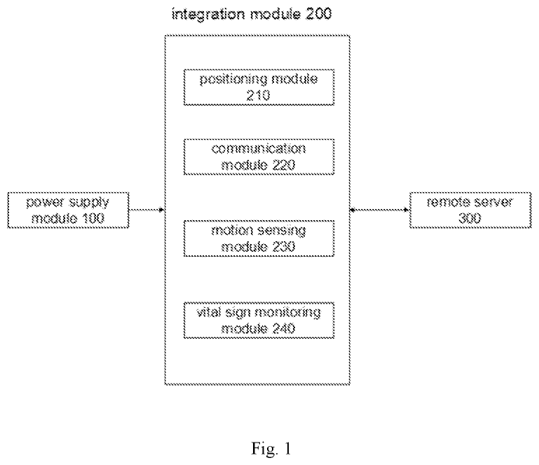

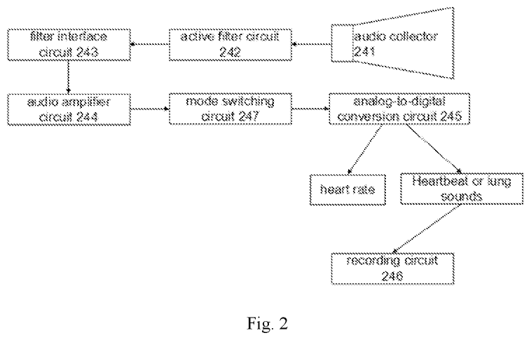

The present invention relates to a system for realizing remote monitoring of the movement health of a pet. As shown in FIG. 1 and FIG. 2, it comprises: a power supply module 100, an integration module 200, a remote server 300 module and a client end.

Said power supply module 100 is electrically connected to said integration module 200. Said remote server 300 module has signal connection to said client end. Said integration module 200 comprises: a positioning module 210, a motion sensing module 230, a vital sign monitoring module 240 and a communication module 220. Said communication module 220 has signal connection to said remote server 300 module. Said positioning module 210 is electrically connected to said communication module 220, wherein said positioning module 210 is used for real-time positioning and path tracking of the pet, and said positioning module 210 is used to send the acquired pet position information and path information to said communication module 220. Said motion sensing module 230 is electrically connected to said communication module 220. Said motion sensing module 230 is used to collect data information on the motion status of the pet, wherein the data information collected is the frequency of the pet's movement over a certain period of time, and to compare said data information with predetermined information to determine the motion status of the pet, wherein the motor state includes sleep state and activity state. Said motion sensing module 230 is also used to send the determined result to said communication module 220, and the communication module 220 sends the result to the client end via the remote 300 server, so that the user can know whether the pet is sleeping or active. Said vital sign monitoring module 240 is electrically connected to said communication module 220, wherein said vital signs monitoring module 240 is used to listen to and acquire pet cardiorespiratory audio and detect pet heart rate and send the acquired cardiorespiratory audio information to said communication module 220, and said communication module 220 sends said cardiorespiratory audio information to said remote server 300 module and performs real-time analysis. The remote server 300 analyzes the obtained cardiopulmonary audio information based on certain pet medical algorithms to determine the health status of the pet. Said remote server 300 module sends the analysis results to said client end for the user to observe the heart and lung sound waveform of the pet in real time, providing physiological parameters such as heart rate, as well as displaying, storing and restoring heart and lung sounds.

Preferably, while listening to and acquiring the heart or lung sounds, the remote server 300 combines the information sent by the positioning module 210 and the motion sensing module 230 to achieve pet motion health monitoring and real-time analysis of the pet's heart and lung sounds during exercise or sleep. The power supply module 100 is a rechargeable lithium battery, so that the device can be used many times. The communication module 220 can use wired communication method or wireless communication method, wherein the wireless communication method can be cellular communication method, WiFi, Bluetooth, UWB, Lora, etc, and the wired communication method can be USB, Type-c, lighting, RS232, IIC, etc. The positioning module 210 is GPS. The motion sensing module 230 measures the pet's motion status by means of a three-axis gyroscope. Said client end is a cell phone or a monitor, so that the customer can easily know the information of the pet. Said device worn by pets is provided with an accommodation section, wherein said accommodation section is provided with an opening. Said integration module 200 and said power supply module 100 are provided in said accommodation section. Said opening is provided with a zipper, thus facilitating the installation or replacement of the integration module 200 and the power supply module 100.

Preferably, said vital sign monitoring module 240 comprises: an audio collector 241, an active filter circuit 242, a filter interface circuit 243, an audio amplifier circuit 244, a recording circuit 246 and an analog-to-digital conversion circuit 245.

Said audio collector 241 is used to receive pet cardiorespiratory audio information. Said audio collector 241 is electrically connected to said active filter circuit 242. Said active filter circuit 242 is electrically connected to said filter interface circuit 243. Said filter interface circuit 243 is electrically connected to said audio amplification circuit 244. Said audio amplification circuit 244 sends the amplified pet cardiorespiratory audio information to the analog-to-digital conversion circuit 245. Said analog-to-digital conversion circuit 245 sends the converted obtained pet cardiorespiratory audio information to said recording circuit 246. Said recording circuit 246 is electrically connected to said communication module 220. Said communication module 220 sends the recorded pet cardiorespiratory audio information to the remote server 300. The remote server 300 thus sends the heart and lung sound information to the client end in the form of tables and ECGs. The remote server 300 also analyzes the obtained heart and lung sound information to derive whether the pet is in a healthy state, and sends the analysis results to the client to facilitate the user to understand the pet health information in more detail. In the present embodiment, the vital sign monitoring module 240 is further provided with a mode switching circuit 247, which is electrically connected to the recording circuit 246. The mode switching circuit 247 is used to switch the object listened to, one mode for listening to heart rate and another mode for listening to lung sounds or heartbeat sounds. Said audio collector 241 is coated with medical silicone so that the device is not too hard and uncomfortable for the pet to wear. The audio collector 241 is a membrane cavity pickup stethoscope head.

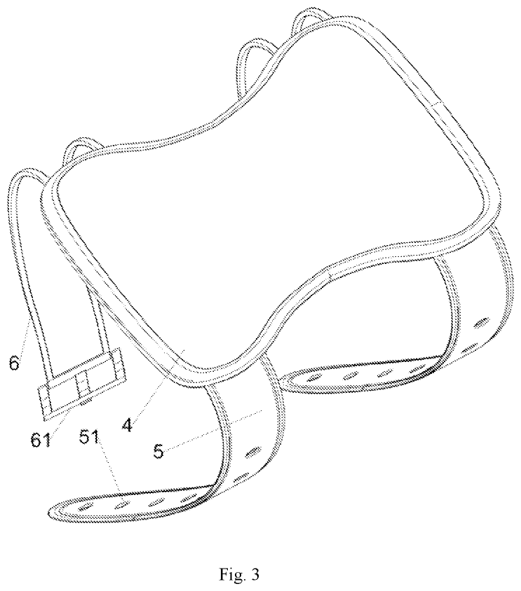

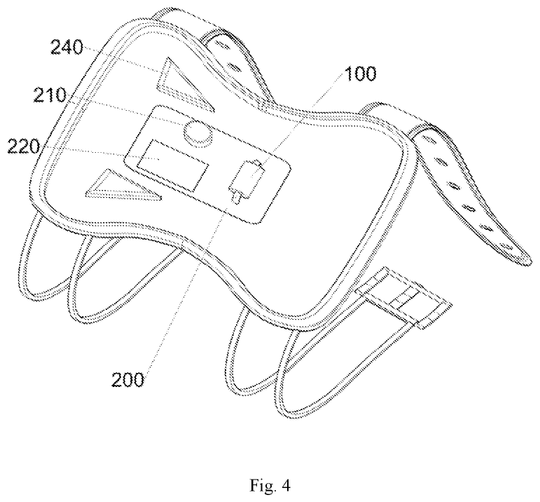

Preferably, as shown in FIG. 3 and FIG. 4, said device worn by pets is further provided with a fixing member and an adjusting member, wherein said fixing member is used to fix said device on the side close to the cardiorespiratory position of the pet and said adjusting member is used to adjust the tightness of said fixing member. In the present invention, said fixing member is a belt and said adjusting member is a buckle (61). The belt includes a first belt and second belt, wherein the first belt is provided with a buckle 61, the second belt is provided with a number of snap holes at different positions. The belt fixes the device worn by pets, whereas the buckle 61 adjusts tightness of the belt, so that the device can be worn comfortably on the pet, and furthermore, the device worn by pets is also suitable for pets with different sizes, in order to improve the adaptability of the device.

The working principle of the present invention is generally as follows: the device worn by pets is brought to the side near the heart and lung position of the pet through the fixing member, the adjusting member is adjusted to make the fixing member and the device fit the pet. The pet wearing device is turned on, the pet wearing device thus locates the pet, monitors the movement status, monitors the vital signs, records the running track, and sends the collected information to the remote server 300 through the communication module 220. The remote server 300 analyzes the collected information, and finally sends the analysis results to the client end, thus enabling the user to understand the health status and movement status of the pet in all aspects. The system for realizing remote monitoring of the movement health of a pet of the present invention effectively solves problems of the existing pet health monitoring equipment, which determines the amount of pet movement only by gyroscopes and can only perform simple monitoring on pet movement and sleeping and cannot truly provide feedback on the pet's vital signs. The present invention achieves a real movement health monitoring function with a reasonable structural improvement and data collection and especially through the pet movement and cardiopulmonary monitoring. In addition, with the improvement of the pet wear module, the present invention achieves a fixed smart device worn by pets, so that the distant pet owners are more at ease, which is conducive to technology promotion and application of the technology.

The above is only to illustrate the technical solution of the present invention and not to limit it. Other modifications or equivalent substitutions made to the technical solution of the present invention by a person of ordinary skill in the art shall be covered by the scope of the claims of the present invention as long as they do not depart from the spirit and scope of the technical solution of the present invention.

What is claimed is:

1. A system for realizing remote monitoring of the movement health of a pet used in a device worn by pets, said system for realizing remote monitoring of the movement health of a pet comprising: an integration module (200), a power supply module (100), a remote server (300) module and a client end, said power supply module (100) being electrically connected to said integration module (200), said remote server (300) module having signal connection to said client end, characterized in that said integration module (200) comprises: a positioning module (210), a motion sensing module (230), a vital sign monitoring module (240) and a communication module (220), wherein
said communication module (220) has signal connection to said remote server (300) module,
said positioning module (210) is electrically connected to said communication module (220), wherein said positioning module (210) is used for real-time positioning and path tracking of the pet, and said positioning module (210) is used to send the acquired pet position information and path information to said communication module (220),
said motion sensing module (230) is electrically connected to said communication module (220), wherein said motion sensing module (230) is used to collect data information on the motion status of the pet and to compare said data information with predetermined information to determine the motion status of the pet, and said motion sensing module (230) is also used to send the determined result to said communication module (220); and
said vital sign monitoring module (240) is electrically connected to said communication module (220), said vital signs monitoring module (240) is used to listen to and acquire pet cardiorespiratory audio and detect pet heart rate and send the acquired cardiorespiratory audio information to said communication module (220), and said communication module (220) sends said cardiorespiratory audio information to said remote server (300) module and performs real-time analysis, and said remote server (300) module sends the analysis results to said client end.

2. The system for realizing remote monitoring of the movement health of a pet according to claim 1, characterized in that said device worn by pets is further provided with a fixing member and an adjusting member, wherein said fixing member is used to fix said device on the side close to the cardiorespiratory position of the pet and said adjusting member is used to adjust the tightness of said fixing member.

3. The system for realizing remote monitoring of the movement health of a pet according to claim 1, characterized in that said communication module (220) can be wired communication means or wireless communication means.

4. The system for realizing remote monitoring of the movement health of a pet according to claim 1, characterized in that said motion sensing module (230) comprises: a three-axis gyroscope, wherein said three-axis gyroscope is used to obtain data information on the motion status of said pet.

5. The system for realizing remote monitoring of the movement health of a pet according to claim 1, characterized in that said vital sign monitoring module (240) comprises: an audio collector (241), an active filter circuit (242), a filter interface circuit (243), an audio amplifier circuit (244), a recording circuit (246) and an analog-to-digital conversion circuit (245), wherein said audio collector (241) is used to receive pet cardiorespiratory audio information, said audio collector (241) is electrically connected to said active filter circuit (242), said active filter circuit (242) is electrically connected to said filter interface circuit (243), said filter interface circuit (243) is electrically connected to said audio amplification circuit (244), said audio amplification circuit (244) sends the amplified pet cardiorespiratory audio information to the analog-to-digital conversion circuit (245), said analog-to-digital conversion circuit (245) sends the converted obtained pet cardiorespiratory audio information to said recording circuit (246), said recording circuit (246) is electrically connected to said communication module (220), said communication module (220) sends the recorded pet cardiorespiratory audio information to the remote server (300).

6. The system for realizing remote monitoring of the movement health of a pet according to claim 5, characterized in that said audio collector (241) is coated with medical silicone.

7. The system for realizing remote monitoring of the movement health of a pet according to claim 5, characterized in that said audio collector (241) is a membrane cavity pickup stethoscope head.

8. The system for realizing remote monitoring of the movement health of a pet according to claim 2, characterized in that said fixing member is a belt and said adjusting member is a buckle (61).

9. The system for realizing remote monitoring of the movement health of a pet according to claim 1, characterized in that said client end is a cell phone or a monitor.

10. The system for realizing remote monitoring of the movement health of a pet according to claim 1, characterized in that said device worn by pets is provided with an accommodation section, said accommodation section is provided with an opening, said integration module (200) and said power supply module (100) are provided in said accommodation section, and said opening is provided with a zipper.
